(19) United States
(12) Patent Application Publication
(10) Pub. No.: US 2006/0171948 A1
(43) Pub. Date: Aug. 3, 2006

(54) Methods of using IL-1 antagonists to reduce C-reactive protein

(76) Inventors: Steven P. Weinstein, Hartsdale, NY (US); Margaret Karow, Putnam Valley, NY (US); Allen Radin, New York, NY (US); William Roberts, Scarsdale, NY (US); Li-Hsien Wang, Somers, NY (US); Scott Mellis, New Rochelle, NY (US)

Correspondence Address: REGENERON PHARMACEUTICALS, INC, 777 OLD SAW MILL RIVER ROAD, TARRYTOWN, NY, 10591, US

(21) Appl. No.: 11/346,007

(22) Filed: Feb. 2, 2006

Related U.S. Application Data
(60) Provisional application No. 60/649,220, filed on Feb. 2, 2005.
(60) Provisional application No. 60/650,339, filed on Feb. 4, 2005.

(51) Int. Cl. A61K 39/395; A61K 38/17

(52) U.S. Cl. 424/145.1; 514/12

(57) ABSTRACT

Methods of reducing C-reactive protein (CRP) in a subject, comprising administering to the subject a therapeutic amount of an interleukin 1 (IL-1) antagonist, wherein CRP is reduced. The IL-1 antagonist is preferably an IL-1-binding fusion protein (IL-1 trap), preferably comprising SEQ ID NO:2.

### CROSS REFERENCE TO RELATED APPLICATIONS

This application claims the benefit under 35 USC §119(e) of U.S. Provisionals 60/649,220 filed 2 Feb. 2005 and 60/650,339 filed 4 Feb. 2005, which applications are herein specifically incorporated by reference in their entirety.

### BACKGROUND

1. Field of the Invention

The invention relates to methods of using interleukin-1 (IL-1) antagonists to reduce C-reactive Protein (CRP) in a subject in need thereof. The invention also relates to methods of reducing the risk of or ameliorating a condition associated with elevated CRP, including, for example, atherosclerosis in a human subject by administrating an IL-1 antagonist. The invention further relates to methods of reducing the risk of adverse events in a subject undergoing or who has undergone a medical procedure such as vein grafts, angioplasty, etc.

2. Description of Related Art

Coronary artery disease (CAD) continues to be a leading cause of death in Western societies, despite major advances in the prevention, detection, and treatment of the disease. Most presentations of acute myocardial ischemia are unheralded—only 20% of acute coronary attacks are preceded by longstanding, stable angina pectoris. Acute coronary syndrome (ACS), an umbrella term used to describe clinical symptoms consistent with unexpected or atypically severe and prolonged acute myocardial ischemia, includes unstable angina (UA) and acute myocardial infarction (AMI). ACS is a major cause of emergency medical care and hospitalization in the U.S.

Methods for identifying patients at risk for acute coronary syndrome by measurement of a serum/plasma marker such as C-reactive protein are known, see for example, U.S. Patent Application 2004/0072805. An in vitro method of screening for modulators of C-reactive protein is described in U.S. Pat. No. 6,764,826. A method for decreasing elevated LDL-cholesterol or LDL-cholesterol/CRP levels is described in U.S. Pat. No. 6,841,547.

Interleukin-1 (IL-1) traps are multimers of fusion proteins containing IL-1 receptor components and a multimerizing component capable of interacting another fusion protein to form a higher order structure, such as a dimer. IL-1 traps are described in WO 00/18932, herein specifically incorporated by reference. The IL-1 trap has been developed as an antagonist of IL-1 in the treatment of rheumatoid arthritis and other inflammatory diseases. A key role of inflammatory mechanisms in the pathogenesis of atherosclerosis and its complications has been recognized and supported by basic and clinical research over the past decade.

### BRIEF SUMMARY OF THE INVENTION

In a first aspect, the invention features a method of reducing C-reactive protein (CRP) in a human subject, comprising administering to the subject an interleukin 1 (IL-1) antagonist such that CRP is reduced. More specifically, the invention features a method of reducing plasma/serum CRP levels in a human subject, comprising administering to the subject an interleukin 1 (IL-1) antagonist such that CRP is reduced relative to pretreatment level.

An IL-1 antagonist is a compound capable of blocking or inhibiting the biological action of IL-1, including fusion proteins capable of trapping IL-1, such as an IL-1 trap. In a preferred embodiment, the IL-1 trap is an IL-1-specific fusion protein comprising two IL-1 receptor components and a multimerizing component, for example, an IL-1 trap described in U.S. patent publication No. 2003/0143697, published 31 Jul. 2003, herein specifically incorporated by reference in its entirety. An IL-1 trap fusion protein comprises an IL-1 binding portion of the extracellular domain of human IL-1 RAcP, an IL-1 binding portion of the extracellular domain of human IL-1 RI, and a multimerizing component. In a specific embodiments, the IL-1 trap is the fusion protein shown in SEQ ID NO:2, encoded by the nucleic acid sequence shown in SEQ ID NO:1. The invention encompasses the use of an IL-1 trap substantially identical to the protein of SEQ ID NO:2, that is, a protein having at least 95% identity, at least 97% identity, at least 98% identity to the protein of SEQ ID NO:2 and capable of binding and inhibiting IL-1. Further, in specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor.

In other embodiments, the methods of the invention may be carried out with an IL-1 antagonist such as a chimeric, humanized or human antibody to IL-1α or β (such as CDP-484, Celltech) or to the IL-1 receptor (for example, AMG-108, Amgen; R-1599, Roche), IL-1 Ra (anakinra, Amgen; IL-1ra gene therapy, Orthogen), and ICE inhibitor, such as Vx-765 (Vertex), p38 MAP inhibitors, IKK 1/2 inhibitors (such as, UK-436303, Pfizer; SPC-839, Serono/Signal), and collagenase inhibitors (PERIOSTAT™, Collagenex).

In specific embodiments, the method of the invention carried out with an IL-1 antagonist which is capable of rapidly reducing CRP levels in a human subject. By “reducing CRP level” is meant a reduction of at least 20%, more preferably at least 30%, even more preferably at least 40%, still more preferably at least 50%, at least 60%, at least 70%, or at least 80% relative to pre-treatment level. Reduction of CRP level may be determined by a reduction relative to pre-treatment level, e.g., reducing CRP level in a subject with elevated CRP level by e.g., 50% relative to pre-treatment level. Achieving a reduction in CRP level in an individual may be determined by, for example, measuring a reduction from pre-treatment level or by achieving a target or normal level with treatment as defined by current medical guidelines.

Reduction in CRP levels achieved rapidly upon administration of said IL-1 antagonist, e.g., within about 1-10 days, preferably in 7 days or less, 6 days or less, 5 days or less, 4 days or less, 3 days or less, 2 days or less, or even within 24 hrs following treatment.

In specific embodiments, a subject suitable for treatment by the method of the invention is a subject at risk for development or recurrence of a condition or an adverse medical event associated with elevated CRP, such as, in non-limiting examples, atherosclerosis, acute coronary syndrome, stroke. In specific embodiments, the subject at risk has an elevated serum CRP level, e.g., as determined by expert consensus guidelines at the time of treatment. In specific embodiments, a person at risk for development or recurrence of a condition associated with elevated CRP has a serum CRP level of ≧1.0 mg/L. In other specific embodiments, a person at risk for development or recurrence of a condition associated with elevated CRP has a serum/plasma CRP level of ≧2.0 mg/L; ≧2.5 mg/L; ≧3.0 mg/L (CDC-AHA guidelines); ≧3.5 mg/L; or ≧4.0 mg/L.

A preferred subject for treatment by the methods of the invention is a subject who will undergo or has undergone a medical procedure associated with risk of adverse medical event, for example, a subject undergoing a revascularization procedure, such as angioplasty, coronary artery bypass graft (CABG), vascular stenting, vein graft, prosthetic graft, or a similar procedure. In specific embodiments, a subject may have a normal serum CRP level or an elevated serum CRP level.

A preferred subject for treatment by the methods of the invention is a subject suffering from or at risk of suffering from a condition which is ameliorated, inhibited, or reduced by a reduction in serum/plasma CRP level.

Conditions characterized by elevated C-reactive protein include, for example, atherosclerosis, including coronary artery disease, cerebral vascular disease, peripheral vascular disease, an infection or an allergic complication resulting from an infection, an inflammatory disease, necrosis, trauma, and/or a malignancy.

The methods of the invention include administration of the IL-1 antagonist by any means known to the art, for example, subcutaneous, intramuscular, intravenous, topical, transdermal or oral routes of administration. Preferably, administration is by subcutaneous or intravenous injection or infusion.

In specific conditions, the method of the invention may encompass a single administration of an IL-1 antagonist, or it may encompass multiple administrations of the IL-1 antagonist. Multiple administrations may include a frequency that is weekly, bi-weekly, monthly, bi-monthly, or quarterly, depending on the condition being treated and/or result desired. In specific embodiments, the IL-1 antagonist is initially administered prior to initiation of a medical procedure such as angioplasty, or initial administration may be simultaneous or following the medical procedure.

The dose of an IL-1 antagonist administered will depend on the condition being treated, the agent utilized, the desired result, and/or the presence of secondary therapeutic agents. When the IL-1 antagonist administered is an IL-trap, for example, as exemplified by the protein of SEQ ID NO:2, a therapeutically effective dose will depend on whether single or multiple doses are given, as well as frequency of administration. Doses may range from about 20 to about 2000 mg protein of IL-1 trap, or 50-2000 mg, 50-500 mg, or 50-350 mg.

In specific embodiments of the therapeutic methods of the invention, the subject is treated with a combination of an IL-1 trap and a second therapeutic agent. The second therapeutic agent may be one or more of a second IL-1 antagonist, such as, for example, anakinra (KINERET®), Amgen), a recombinant, nonglycosylated form of the human IL-1 receptor antagonist (IL1Ra), or an anti-IL-18 drug such as IL-18BP or a derivative, an IL-18-binding fusion protein (IL-18 “trap”), anti-IL-18, anti-IL-18R1, or anti-IL-18RAcP. Other co-therapies include low dose colchicine for FMF, anti-platelet agents (such as aspirin or clopidogrel (PLAVIX™, Sanofi-Aventis) or other NSAIDs, anti-ischemic (such as nitroglycerin or beta blockers), anti-thrombin such as heparin, hirudin, bivlarudin, fibrinolytic agents, GPIIb/IIIa antagonists (abciximab, eptifibatide, tirofiban) steroids such as prednisone, prednisolone, methotrexate, low dose cyclosporine A, folate, TNF inhibitors such as etanercept (ENBREL®), or adalimubab (HUMIRA®), other inflammatory inhibitors such as inhibitors of caspase-1, p38 MAP kinase, IKK1/2, CTLA-4lg, anti-IL-6 or anti-IL6Ra, etc. A second agent may include a cholesterol-lowering agent such as hydroxymethylglutaryl-CoA reductase inhibitors (statins), vitamin E and derivatives thereof, and fish oil (Chan et al. (2002) supra). Further, secondary agents may include insulin sensitizers. The method of the invention may also be combined with lifestyle changes to reduce risk of development or recurrence of an undesirable conditions, for example, reduction of CRP may be achieved with a combination of an IL-1 antagonist and exercise, weight loss, reduction of alcohol intake, or improved control of a condition such as diabetes.

In a more specific aspect, the invention features a method of reducing C-reactive protein (CRP) in a human subject, comprising administering to the subject an interleukin 1 (IL-1) antagonist such that CRP is reduced, wherein the IL-1 antagonist is an IL-1-specific fusion protein (IL-1 “trap”) as described above.

In a second aspect, the invention features a method of treating, inhibiting, or ameliorating or reducing the risk of suffering from atherosclerosis, comprising administering to a subject in need an interleukin 1 (IL-1) antagonist. In specific embodiments, atherosclerosis includes coronary artery disease, cerebral vascular disease, and/or peripheral vascular disease.

In a third aspect, the invention features a method of treating, inhibiting, or ameliorating or reducing the risk of development or recurrence of acute coronary syndrome (ACS), comprising administering to a subject in need an interleukin 1 (IL-1) antagonist.

In a fourth aspect, the invention features a method of treating, inhibiting, or ameliorating angina, comprising administering to a subject in need an interleukin 1 (IL-1) antagonist.

In a fifth aspect, the invention features a method of treating, inhibiting, or ameliorating or reducing the risk of suffering a myocardial infarction, comprising administering to a subject in need an interleukin 1 (IL-1) antagonist. The myocardial infarction may be non-ST-segment elevation myocardial infarction (NSTEMI), ST-segment elevation myocardial infarction (STEMI), Q-wave elevation myocardial infarction, non-Q-wave elevation myocardial infarction.

Other objects and advantages will become apparent from a review of the ensuing detailed description.

### BRIEF DESCRIPTION OF THE FIGURES

FIG. 1 is a graph showing median percent change from baseline CRP in RA subjects treated with placebo (♦), 25 mg (□), 50 mg (▴), or 100 (x) mg IL-1 trap.

FIG. 2 is a graph showing the mean (open) and median (solid) decrease in CRP of subjects treated with IL-1 trap at day 6. Subjects are grouped as follows: 0=placebo, 50 mg, 80-104 mg, and 120-2000 mg.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

C-Reactive Protein and Related to Inflammatory Diseases

Atherosclerosis is recognized as a chronic inflammatory process (Ross (1999) Am Heart J. 138:S419-20). Studies have shown that increased plasma concentrations of high-sensitivity C-reactive protein (hs-CRP or CRP), a sensitive marker for low-grade inflammation, are associated with increased risk of cardiovascular events (Rifai et al. (2001) Clin Chem 47:403-11; Taubes (2002) Science 296:242-245). CRP can be measured in the serum or plasma. CRP is released by the body in response to acute injury, infection or other inflammation-inducing conditions, such as atherosclerosis. Increasing evidence suggests that increased CRP concentrations are also associated with obesity and a cluster of metabolic risk factors related to visceral obesity, such as hyperinsulinemia, hypertriglycridemia, and low high density lipoprotein (HDL) (see, for example, Hak et al. (1999) Arterioscler Thromb Vasc Biol 19:1986-91; Chambers et al. (2001) Circulation 104:145-150; Chan et al. (2002) Clin Chem 48:877-883). Agents with broad or undefined mechanisms which reduce CRP have been described (for example, Vx-702, Vertex).

Cholesterol can combine with low density lipoprotein (LDL) to form low density lipoprotein-cholesterol. Too much cholesterol or LDL-cholesterol in the bloodstream is typically a major risk factor for cardiovascular disease. For example, excessive cholesterol can lead to formation of atheroscleromatous plaques. These plaques can cause narrowing and hardening of the arteries (i.e., atherosclerosis), which can impede blood flow and lead to a heart attack or stroke. In addition, relatively small atheroscleromatous plaques can become destabilized due to, for example, degradation of the connective tissue (i.e., collagen) “cap.” Destabilization of the plaques can result in rupture of the plaque and thrombosis, which can lead to myocardial infarction. Current treatment for lowering cholesterol, LDL-cholesterol, or CRP includes a class of drugs known as statins. Statins generally alter the metabolism of various constituents within the cholesterol metabolic pathway. However, statins are associated with numerous side effects, including elevation of plasma triglycerides, increased liver aminotransferase activity, abdominal discomfort, nausea, vomiting, diarrhea, malaise, QT interval prolongation, decreased high-density lipoprotein levels, and risk of rhabdomyolysis.

Elevated CRP levels are also associated with a wide variety of diseases, including, for example, chemodialysis, surgery, sickle cell anemia, diabetes, obesity, hypofibrinolysis, multiple organ failure, and heart transplant.

Acute Coronary Syndrome (ACS) and Related Conditions

Although detailed practice guidelines for stable angina and ACS sub-categories have been published recently by task forces of the American Heart Association/American College of Cardiology, management and treatment strategies for these diagnostic categories are dynamic. Decisions regarding medical and interventional treatments are typically based on the diagnostic category (stable angina, UA, NSTEMI, STEMI), other patient-specific findings, and emerging data and treatment modalities.

Treatment strategies in patients with stable angina include lifestyle modification and drug therapy for risk factor modification (obesity, tobacco use, dyslipidemia, hypertension, metabolic syndrome), ACE inhibition, beta-adrenergic receptor blockade, aspirin, and angiographically directed revascularization. Treatment strategies in UA/NSTEMI include antiplatelet therapies such as aspirin, thienopyridine (ticlopidine, clopidogrel, platelet glycoprotein IIIb/IIa antagonists), anticoagulants, lipid risk factor modification, and angiographically directed revascularization. Both clopidogrel and abciximab modestly suppress the rise in circulating inflammatory markers after PCI.

Autoinflammatory Diseases

Autoinflammatory diseases are illnesses characterized by episodes of inflammation that, unlike autoimmune disorders, lack the production of high titer autoantibodies or antigen-specific T cells. There is growing genetic and clinical evidence that IL-1 plays a pathogenic role in several of these diseases. Autoinflammatory disorders include, for example, Neonatal Onset Multisystem Inflammatory Disease (NOMID), Muckle-Wells Syndrome (MWS), and Familial Cold Autoinflammatory Syndrome (FCAS), Familial Mediterranean Fever (FMF), and adult Still's disease. FMF is associated with mutations in pyrin encoding MEFV. NOMID, MWS and FCAS are associated with mutations in cryopyrin-encoding CIAS1.

Coronary Interventions

The methods of the invention are advantageously used with a subject who is to undergo or has undergone a medical procedure such as angioplasty, vascular stenting, vein graft (such as CABG or peripheral vein graft), dialysis, etc. Many of these procedures are known to cause an increase or a further increase in CRP levels, and are believed to place the subject at risk for an adverse medical event, e.g., such as a myocardial infarction.

Definitions

By the term “blocker”, “inhibitor”, or “antagonist” is meant a substance that retards or prevents a chemical or physiological reaction or response. Common blockers or inhibitors include but are not limited to antisense molecules, antibodies, antagonists and their derivatives. More specifically, an example of an IL-1 blocker or inhibitor is an IL-1 antagonist including, but not limited to, an IL-1-binding fusion protein (termed and IL-1 “trap”) which binds and inhibits IL-1.

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “substantially identical” is meant a protein sequence having at least 95% identity to an amino acid sequence of SEQ ID NO:2, and capable of binding IL-1 and inhibiting the biological activity of IL-1.

The term “identity” or “homology” is construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions will be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

IL-1 Trap and IL-1 Antagonists

Interleukin-1 (IL-1) traps are multimers of fusion proteins containing IL-1 receptor components and a multimerizing component capable of interacting with the multimerizing component present in another fusion protein to form a higher order structure, such as a dimer. Cytokine traps are a novel extension of the receptor-Fc fusion concept in that they include two distinct receptor components that bind a single cytokine, resulting in the generation of antagonists with dramatically increased affinity over that offered by single component reagents. In fact, the cytokine traps that are described herein are among the most potent cytokine blockers ever described. Briefly, the cytokine traps called IL-1 traps are comprised of the extracellular domain of human IL-1 R Type I (IL-1 RI) or Type II (IL-1 RII) followed by the extracellular domain of human IL-1 Accessory protein (IL-1AcP), followed by a multimerizing component. In a preferred embodiment, the multimerizing component is an immunoglobulin-derived domain, such as, for example, the Fc region of human IgG, including part of the hinge region, the CH2 and CH3 domains. An immunoglobulin-derived domain may be selected from any of the major classes of immunoglobulins, including IgA, IgD, IgE, IgG and IgM, and any subclass or isotype, e.g. IgG1, IgG2, IgG3 and IgG4; IgA-1 and IgA-2. Alternatively, the IL-1 traps are comprised of the extracellular domain of human IL-1AcP, followed by the extracellular domain of human IL-1 RI or IL-1RII, followed by a multimerizing component. For a more detailed description of the IL-1 traps, see WO 00/18932, which publication is herein specifically incorporated by reference in its entirety. Preferably, the IL-1 trap is the amino acid sequence shown in SEQ ID NO:2, or a substantially identical protein at least 95% identity to a sequence of SEQ ID NO:2 and capable of binding and inhibiting IL1.

In specific embodiments, the IL-1 antagonist comprises an antibody fragment capable of binding IL-1α, IL-1β, IL-1R1 and/or IL-1RAcp, or a fragment thereof. The preferred embodiment is an antagonist of IL-1β. One embodiment of an IL-1 antagonist comprising one or more antibody fragments, for example, single chain Fv (scFv), is described in U.S. Pat. No. 6,472,179, which publication is herein specifically incorporated by reference in its entirety. In all of the IL-1 antagonist embodiments comprising one or more antibody-derived components specific for IL-1 or an IL-1 receptor, the components may be arranged in a variety of configurations, e.g., a IL-1 receptor component(s)-scFv(s)-multimerizing component; IL-1 receptor component(s)-multimerizing component-scFv(s); scFv(s)-IL-1 receptor component(s)-multimerizing component, ScFv-ScFv-Fc, etc., so long as the molecule or multimer is capable of inhibiting the biological activity of IL-1.

Treatment Population

In specific embodiments, populations of human subjects which are preferably treated by the methods of the invention include a subject determined to be at risk for development or recurrence of a condition or adverse medical event, which condition may be ameliorated, reduced or inhibited by reduction of serum/plasma CRP levels. In specific embodiments, a subject preferably treated by the methods of the invention is a subject exhibiting an elevated serum/plasma CRP level which places that subject at risk for development or recurrence of a condition associated with elevated serum/plasma CRP levels. In specific embodiments, the subject to be treated by the methods of the invention is a subject who is to undergo or has undergone a medical procedure associated with risk of an adverse medical event, such as, in non-limiting examples, may be a medical procedure such as angioplasty, CABG, stenting, vein graft, or a similar procedure.

In specific embodiments, the subject being treated is a human diagnosed as suffering from or at risk for suffering from a condition characterized by elevated C-reactive protein (Pepys et al. (2003) J Clin Invest 111:1805-1812). For example, atherosclerosis, including coronary artery disease, cerebral vascular disease, pheripheral vascular disease, an infection or an allergic complication resulting from an infection, an inflammatory disease, necrosis, trauma, and/or a malignancy. Coronary artery disease includes such conditions as, for example, acute coronary syndrome (ACS), myocardial ischemia, unstable angina, acute myocardial infarction, ST elevation myocardial infarction, non-ST elevation myocardial infarction, Q-wave myocardial infarction, non-Q-wave myocardial infarction. An infection may be bacterial, systemic or severe fungal, mycobacterial, or viral. Allergic complications resulting from infection include, for example, rheumatic fever or erythema nodosum. Inflammatory diseases include rheumatoid arthritis, juvenile chronic arthritis, ankylosing spondylitis, psoriatic arthritis, systemic vasculitis, polymyalgia rheumatica, Reiter disease, Crohn's disease, and/or Familial Mediterranean disease. Necrosis includes myocardial infarction, tumor embolization, and acute pancreatitis. Trauma may result from surgery, burns and/or fractures. Malignancy includes lymphoma, carcinoma and sarcoma.

Combination Therapies

In numerous embodiments, the IL-1 antagonists of the present invention may be administered in combination with one or more additional compounds or therapies. Combination therapy may be simultaneous or sequential. An IL-1 antagonist may be combined with, for example, TNF-inhibiting agents such as etanercept (ENBREL®, Amgen), infliximab (REMICADE®, Centocor), adalimuab (HUMIRA®, Abbott), thalidomide and thalidomide-related compounds, steroids, anakinra (KINARET®), Amgen), colchicine, methotrexate, cyclosporine, chlorambucil, cyclophosphamide, prednisolone, anti-IL-6 or anti-IL6Ra, and sulfasalazine. The IL-1 antagonist may also be combined with HMG-CoA reductase inhibitors, such as LESCOL™ (Novartis), LIPITOR™ (Pfizer), MEVACOR™ (Merck), PRAVACHOL™ (Bristol Myers Squibb), ZOCOR™ (Merck) or anti-lipidemic agents such as, COLESTID™ (Pfizer), WELCHOL™ (Sankyo), ATROMID-S™ (Wyeth), LOPID™ (Pfizer), TROCOR™ (Abbott), PPARα agonists, thiazolidinediones such as PPARγ and/or PPARδ agonists, and mixtures thereof.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Methods of Administration

The invention provides methods of treatment comprising administering to a subject a therapeutically effective amount of an IL-1 antagonist. In a preferred aspect, the agent is substantially purified (e.g., substantially free from substances that limit its effect or produce undesired side-effects).

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### A Randomized, Double-Blind, Placebo-Controlled Dose Ranging Study of IL-1 trap in Patients with Active Rheumatoid Arthritis

A study was conducted to compare weekly subcutaneous doses of 25, 50, and 100 mg of the IL-1 antagonist of SEQ ID NO:2 (“IL-1 trap”) with placebo in 201 patients with rheumatoid arthritis (RA), of which 114 (56.7%) completed the study. At baseline, patients were required to have a CRP level greater than 3.0 mg/L (0.3 mg/dL). CRP levels were measured at weeks 2, 3, 4, 6, 8, 10, and 12. The C-reactive protein assay was performed by immunonephelometry (Dade Behring nephelometer). Polystyrene particles coated with monoclonal antibodies to CRP were agglutinated when mixed with samples containing CRP. The intensity of the scattered light in the nephelometer depends on the CRP content of the sample and the CRP concentrations are determined versus dilutions of a standard of a known concentration.

Eligible patients were males or females between 18 and 75 years of age (inclusive) who had a diagnosis of RA, with disease duration of no less than 6 months. Patients had to have at least 10 swollen and 10 tender joints (58/60 joint count) upon entry into the study at screening and at baseline, a CRP ≧0.3 mg/dL, and had incomplete responses to conventional DMARD therapy. IL-1 trap (SEQ ID NO:2) was provided in sealed, sterile, single-use 3-mL vials containing 1.2 mL of IL-1 trap at a concentration of 12.5, 25, or 50 mg/mL in a solution of 150 mM sodium chloride, 0.2% polysorbate 20, 5 mM sodium citrate, 5 mM sodium phosphate, and 20% sucrose, at a pH of 6. IL-1 trap was administered as a 1.0-mL subcutaneous (SC) injection alternating among the deltoid area, abdomen, and anterior thighs. Placebo consisted of vehicle solution (150 mM sodium chloride, 0.2% polysorbate 20, 5 mM sodium citrate, 5 mM sodium phosphate, and 20% sucrose, at a pH of 6) provided in identical vials and administered as a 1.0-mL SC injection alternating among the deltoid area, abdomen, and anterior thighs. Each patient was to receive 12 SC doses of study drug or placebo over 12 weeks.

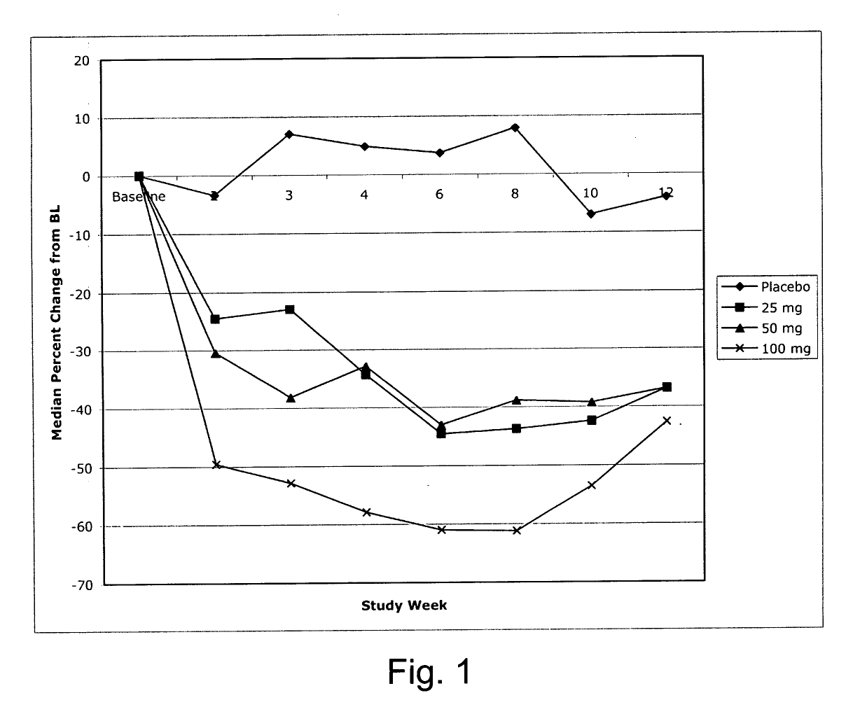

Results. Mean change in CRP (mg/dL) was 0.072 for the placebo group, −0.675 for the 25 mg group, −1.021 for the 50 mg group, and −1.363 for the 100 mg group. FIG. 1 is a graph of the median percentage change from baseline serum CRP values over time.

### Example 2

### Effect of Two Formulations of IL-1 Trap on Serum CRP levels in Volunteers

Study design. A study was conducted to determine the effect on serum CRP of two formulations of IL-1 trap injected in a range of volumes subcutaneously to normal volunteers. A six-week, double blind, placebo-controlled, single dose, single center study with four sequential dose groups: Group A: 1:1:1:1:1:1 balanced random allocation; Group B: A single two-injection dose (1.5 mL per injection); Group C: A single two-injection dose (2.0 mL per injection); and Group D: A single two-injection dose (2.0 mL per injection). There were 12 subjects in each of 6 treatment groups, for a total of 72 subjects in Group A, a total of 7 subjects in Group B, 14 subjects in Group C, and 7 subjects in Group D.

Inclusion requirements. Normal volunteers aged 18-70 with no known significant concomitant illness and no concomitant medication use except as-needed analgesics (at protocol specified times) and/or oral contraceptive pills.

IL-1 trap formulations and placebo. All test materials were injected sub-cutaneously or intravenously. The IL-1 trap (SEQ ID NO:2) was administered as single dose subcutaneously of 50 mg, 80 mg, 104 mg, 120 mg, 160 mg, 240 mg, 320 mg of the IL-1 trap. 300, 500, 1000, and 2000 mg was administered as an intravenous dose. Placebo was administered as either a subcutaneous or intravenous dose.

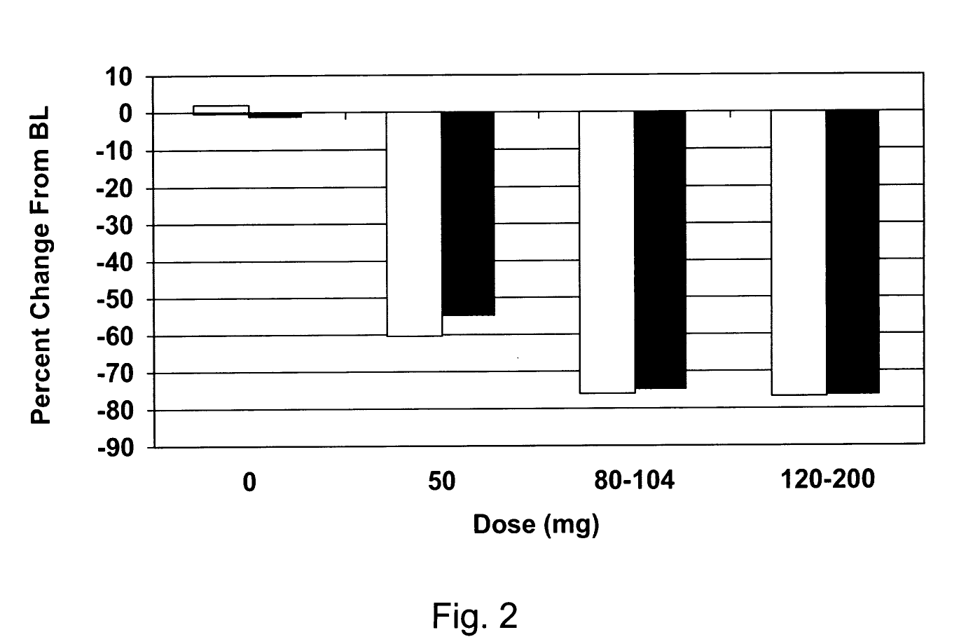

Results. FIG. 2 is a graph showing the mean and median decrease in CRP of subjects treated with IL-1 trap at day 6. The results are grouped as 0=placebo, 50 mg, 80-104 mg, and 120-2000=120, 160, 240, 2000 mg). Table 1 shows the effect of treatment with the IL-1 trap (combined results for all dosage groups 50-2000 mg) on serum CRP levels by weight of subject for those subject with have a post-treatment CRP level of at least 2.37 mg/dL. Regardless of weight or baseline CRP level, IL-1 trap was observed to significantly reduce CRP levels 68-78% from baseline.

TABLE 1
IL-1 Trap Reduction of Serum CRP by Weight Group at Day 6

| | | % Change for Subjects |
|---|---|---|
| Weight Group (kg) | Treatment | with CRP ≧ 2.87 mg/dL |
| Wt ≦ 68 | Placebo | +7.73% (n = 1) |
| | IL-1 trap | −78.52% (n = 2) |
| 68 < wt ≦ 80.1 | Placebo | +6.66 (n = 2) |
| | IL-1 trap | −74.72% (5) |
| wt > 80.1 | Placebo | −3.19% (3) |
| | IL-1 trap | −68.27% (10) |

### Example 3

### Effect of IL-1 Trap on Serum CRP on Adults with Autoinflammatory Disease

In this study, IL-1 trap was administered as a 3 100 mg subcutaneous doses given in 3 consecutive days (days 1-3) to subjects with clinically active autoinflammatory disorders, including the CIAS1-associated disorders, FMF, and adult-onset Still's disease. CRP (mg/dL) was measured as described above on one or more of days 0-29 after administration of the trap. Baseline values for CRP and CRP levels four days after the last 100 mg dose are shown in Table 2. Significant decreases in CRP levels were achieved within four days of administration.

TABLE 2
Effect of IL-1 Trap on CRP levels in Subjects
with Autoinflammatory Disease

CRP (mg/dL)

| Subject | Baseline | Day 4 |
|---|---|---|
| 1 | 3.18 | 0.68 |
| 2 | 8.59 | 1.3 |
| 3 | 3.38 | 0.10 |

What is claimed is:

1. A method of reducing C-reactive protein (CRP) in a human subject, comprising administering to the subject an interleukin 1 (IL-1) antagonist such that CRP is reduced.

2. The method of claim 1, wherein the IL-1 antagonist is an IL-1-binding fusion protein, an antibody specific for IL-1, an antibody specific for an IL-1α or IL-1β receptor, IL-1Ra, or an ICE inhibitor.

3. The method of claim 2, wherein the IL-1-binding fusion protein is the protein of SEQ ID NO:2, or a protein having at least 95% identity to the protein of SEQ ID NO:2 and capable of binding and inhibiting IL-1.

4. The method of claim 1, wherein CRP is reduced by at least 20% relative to pre-treatment level.

5. The method of claim 1, wherein CRP is reduced within 1-14 days following treatment.

6. The method of claim 1, wherein the subject is selected from one or more of the following groups:
(i) the subject is at risk for development or recurrence of a condition associated with elevated CRP;
(ii) the subject will undergo or has undergone a medical procedure associated with risk of adverse medical event;
(iii) the subject is determined to have an elevated CRP level relative to a normal subject; and
(iv) the subject suffers from a condition which is ameliorated by a reduction in CRP.

7. The method of claim 6, wherein the subject is at risk for development or occurrence of atherosclerosis, acute coronary syndrome, and/or stroke.

8. The method of claim 6, wherein an elevated CRP ≧1.0 mg/L.

9. The method of claim 6, wherein the medical procedure is a revascularization procedure.

10. The method of claim 9, wherein the revascularization procedure is angioplasty, coronary artery bypass graft (CABG), stenting, or vein graft.

11. The method of claim 6, wherein the condition associated with an elevated CRP is atherosclerosis or obesity.

12. The method of claim 11, wherein atherosclerosis includes coronary artery disease, cerebral vascular disease, peripheral vascular disease, an infection or an allergic complication resulting from an infection, an inflammatory disease, necrosis, trauma, and/or a malignancy.

13. The method of claim 1, wherein administration is subcutaneous or intravenous injection or infusion.

14. The method of claim 13, wherein administration is a single or multiple doses.

15. The method of claim 14, wherein multiple doses are administered weekly, bi-weekly, monthly, bi-monthly, or quarterly.

16. The method of claim 15, wherein the IL-1 antagonist is administered prior to, simultaneously, or following a medical procedure.

17. The method of claim 12, wherein the IL-1 trap is administered in a range of about 20 to 2000 mg protein.

18. The method of claim 1, further comprising administering a second therapeutic agent.

19. The method of claim 18, wherein the second therapeutic agent is one or more agent(s) selected from the group consisting of anakinra, IL1Ra, an anti-IL-18 agent, colchicine, an anti-platelet agent. an anti-ischemic agent, an anti-thrombin agent, a GPIIb/IIIIa antagonist, a steroid, methotrexate, cyclosporine A, folate, a TNF inhibitor, a caspase-1 inhibitor, a p38 MAP inhibitor, an IKK1/2 inhibitor, a CTLA-4lg inhibitor, an IL-6 inhibitor, a cholesterol-lowering agent, a statin, fish oil, and an insulin sensitizer, or a combination thereof.


SEQUENCE LISTING (2 sequences)

SEQ ID NO: 1 - DNA, 2703 bp, Artificial Sequence
  Synthetic
atggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaaaaatgcaaggaacgtgaagaaaaaataattttagtgagctcagcaaatgaaatcgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 900 aa, Artificial Sequence
  Synthetic
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK